(19) United States
(12) Patent Application Publication
(10) Pub. No.: US 2010/0234358 A1
(43) Pub. Date: Sep. 16, 2010

(54) NERAMEXANE FOR THE TREATMENT OF NYSTAGMUS

(75) Inventors: Angelika Hanschmann, Frankfurt Am Main (DE); Michael Althaus, Offenbach (DE); Albrecht Stoffler, Frankfurt Am Main (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO. KGAA, Frankfurt Am Main (DE)

(21) Appl. No.: 12/451,950

(22) PCT Filed: Jun. 6, 2008
(86) PCT No.: PCT/EP2008/004531

§ 371 Date: May 24, 2010

Related U.S. Application Data
(60) Provisional application No. 60/933,808, filed on Jun. 8, 2007.

(30) Foreign Application Priority Data
Jun. 8, 2007 (EP) 07252336.8
Apr. 7, 2008 (EP) 08006925.5

(51) Int. Cl. A61K 31/5517; A61K 31/13; A61K 31/195; A61K 31/407; A61K 31/44; A61P 25/00

(52) U.S. Cl. 514/220; 514/579; 514/561; 514/567; 514/411; 514/352

(57) ABSTRACT

The present invention relates to the treatment of an individual diagnosed with nystagmus comprising administering to the individual an effective amount of a 1-aminoalkylcyclohexane derivative, for example neramexane or a pharmaceutically acceptable salt thereof, such as neramexane mesylate.

### FIELD OF THE INVENTION

The present invention relates to the treatment of an individual diagnosed with nystagmus comprising administering to the individual an effective amount of a 1-aminoalkylcyclohexane derivative, such as neramexane or a pharmaceutically acceptable salt thereof, such as neramexane mesylate.

### BACKGROUND OF THE INVENTION

This invention relates to methods of treating patients suffering from nystagmus. Nystagmus is defined as repetitive to and fro involuntary eye movements (i.e. oscillations). These oscillations may be horizontal, vertical, or torsional, or any combination of the three. Nystagmus is also sometimes referred to as ocular tremor or oscillopsia.

To date, over forty subtypes of nystagmus have been classified. Nystagmus may be in congenital or acquired forms, and may be further classified based on the direction and/or velocity of movements or the underlying diseases. Infantile or congenital nystagmus may be either idiopathic or associated with ocular disorders (such as retinal diseases, including albinism, retinal detachment, early congenital cataract or glaucoma, and optic nerve abnormalities). Acquired nystagmus is associated with numerous neurological, opthalmologic or metabolic/toxic disorders (such as Multiple Sclerosis, stroke, head trauma, vestibular disturbances, brain tumors, Ménière's disease, Wernicke-Korsakoff syndrome, encephalopathy, lateral medullary syndrome, aniridia, optic nerve hypoplasia, Noonan syndrome, and Pelizaeus-Merzbacher syndrome). Nystagmus includes downbeat nystagmus, upbeat nystagmus, seesaw nystagmus, periodic alternating nystagmus, and acquired pendular nystagmus.

There is currently no approved drug for the treatment of nystagmus. A published investigation (Choudhuri, et al., Eye, 2006) of the pharmacological management of acquired nystagmus among UK neurologists and ophthalmologists revealed that baclofen and gabapentin are used by far most frequently, followed by clonazepam and a variety of other drugs, including carbamazepine, benzhexyl, ondansetrone, buspirone, memantine, and botulinum toxin; however, the majority of physicians estimated the average treatment effect of gabapentin and baclofen as 25% or less improvement in visual acuity and only a few rated the symptomatic improvement of nystagmus higher than 25%. Thus, a need exists for improved drug therapy for nystagmus.

Neramexane, also known as 1-amino-1,3,3,5,5-pentamethylcyclohexane, is a member of the class of orally active 1-aminoalkylcyclohexanes, and has been found to be useful in the therapy of various diseases especially in certain neurological diseases, including Alzheimer's disease and neuropathic pain. Neramexane and its derivatives are disclosed in detail in U.S. Pat. Nos. 6,034,134 and 6,071,966, the subject matter of which patents is hereby incorporated by reference. It is believed that the therapeutic action of neramexane is related to the inhibition of the effects of excessive glutamate at the N-methyl-D-aspartate (NMDA) receptors of nerve cells, for which reason the compound is also categorized as an NMDA antagonist, or NMDA receptor antagonist. More specifically, neramexane appears to be a low to moderate-affinity, non-competitive NMDA-receptor antagonist believed to selectively block the excitotoxic effects associated with abnormal transmission of glutamate.

There have been no clinical studies with neramexane in patients diagnosed with nystagmus according to published reports. The NMDA receptor antagonist memantine has demonstrated activity in acquired pendular nystagmus due to multiple sclerosis (Starck, et al., J. Neurol., 1997, 244, 9-16 and Starck, et al., J. Neurol., 1999, 246 (Suppl. 1), 41), and a recent clinical study (McLean, et al., Ann. Neurol., 2007, 61, 130-138) also demonstrated that the NMDA receptor antagonist memantine may be effective in the treatment of congenital idiopathic and acquired nystagmus.

### SUMMARY OF THE INVENTION

The present invention relates to the treatment of an individual diagnosed with nystagmus, comprising administering to the individual an effective amount of a 1-aminoalkylcyclohexane, such as neramexane or a pharmaceutically acceptable salt thereof, such as neramexane mesylate.

A further aspect of the invention relates to the use of a 1-aminoalkylcyclohexane derivative, such as neramexane or a pharmaceutically acceptable salt thereof, such as neramexane mesylate, for the manufacture of a medicament for treatment of an individual diagnosed with nystagmus.

A further aspect of the invention relates to a pharmaceutical composition for the treatment of nystagmus comprising a therapeutically effective amount of a 1-aminoalkylcyclohexane derivative, such as neramexane or a pharmaceutically acceptable salt thereof, such as neramexane mesylate, and at least one pharmaceutically acceptable carrier or excipient.

A further aspect of the invention relates to a pharmaceutical composition for the treatment of nystagmus comprising a therapeutically effective amount of a 1-amino-alkylcyclohexane derivative, such as neramexane or a pharmaceutically acceptable salt thereof, such as neramexane mesylate, in an immediate or modified release formulation.

A further aspect of the invention relates to the treatment of an individual diagnosed with nystagmus comprising administering to the individual a 1-amino-alkylcyclohexane derivative, such as neramexane or a pharmaceutically acceptable salt thereof, such as neramexane mesylate, and an additional pharmaceutical agent, which has been shown to be effective in treating nystagmus.

A further aspect of the invention relates to the treatment of an individual diagnosed with nystagmus comprising administering to the individual a 1-aminoalkylcyclohexane derivative, such as neramexane or a pharmaceutically acceptable salt thereof, such as neramexane mesylate, and a pharmaceutical agent selected from memantine, gabapentin, vigabatrin, pregabalin, 4-aminopyridine, 3,4-diaminopyridine, baclofen, scopolamine, and clonazepam.

A further aspect of the invention relates to a pharmaceutical composition comprising a therapeutically effective amount of a 1-aminoalkylcyclohexane derivative, such as neramexane or a pharmaceutically acceptable salt thereof, such as neramexane mesylate, in combination with other therapies for nystagmus and, optionally, at least one pharmaceutically acceptable carrier or excipient.

A further aspect of the invention relates to a pharmaceutical composition comprising a therapeutically effective amount of a 1-aminoalkylcyclohexane derivative, such as neramexane or a pharmaceutically acceptable salt thereof, such as neramexane mesylate, in combination with an additional pharmaceutical agent which has been shown to be effective for the treatment of nystagmus and, optionally, at least one pharmaceutically acceptable carrier or excipient.

A further aspect of the invention relates to a pharmaceutical composition comprising a therapeutically effective amount of a 1-aminoalkylcyclohexane derivative, such as neramexane or a pharmaceutically acceptable salt thereof, such as neramexane mesylate, in combination with an therapeutically effective amount of an additional pharmaceutical agent which has been shown to be effective for the treatment of nystagmus and, optionally, at least one pharmaceutically acceptable carrier or excipient.

A further aspect of the invention includes a pharmaceutical composition comprising a therapeutically effective amount of a 1-aminoalkylcyclohexane derivative, such as neramexane or a pharmaceutically acceptable salt thereof, such as neramexane mesylate, a pharmaceutical agent selected from memantine, gabapentin, baclofen, vigabatrin, pregabalin, 4-aminopyridine, 3,4-diaminopyridine, scopolamine, and clonazepam, and at least one pharmaceutically acceptable carrier or excipient.

A further aspect of the invention includes a pharmaceutical composition comprising a therapeutically effective amount of a 1-aminoalkylcyclohexane derivative, such as neramexane or a pharmaceutically acceptable salt thereof, such as neramexane mesylate, and a pharmaceutical agent selected from memantine, gabapentin, vigabatrin, pregabalin, 4-aminopyridine, 3,4-diaminopyridine, baclofen, scopolamine and clonazepam, in an immediate or a modified release formulation.

In a further aspect of the invention, i.e. within the treatment of nystagmus, a therapeutically effective amount of the 1-aminoalkylcyclohexane derivative such as neramexane or a pharmaceutically acceptable salt thereof, such as neramexane mesylate, is administered via an interval therapy, i.e. in its broadest aspect, is administered daily for a first period of at least three (3) months, followed by a second period of at least one (1) month wherein the 1-aminoalkylcyclohexane derivative, such as neramexane or a pharmaceutically acceptable salt thereof, such as neramexane mesylate, is administered at a dose which is 0-75% of the therapeutically effective dose.

The 1-aminoalkylcyclohexane derivative such as neramexane or a pharmaceutically acceptable salt thereof, such as neramexane mesylate, as well as the medicament specified herein may be administered according to the above discussed administration scheme. In one embodiment the derivative/medicament is specifically adapted to provide the respective information regarding the administration scheme to the patient. The respective information regarding the specific administration scheme may be provided via e.g. the respective information in or on the package, the dosage form, such as the appearance thereof, e.g. via tablet color or tablet form, and/or the package leaflet and/or the patient information.

In a further aspect of the invention, the therapeutically effective amount of the 1-aminoalkylcyclohexane derivative such as neramexane or a pharmaceutically acceptable salt thereof, such as neramexane mesylate, is administered daily for a period of at least three (3) months, followed by a period of at least one month wherein the 1-aminoalkylcyclohexane derivative is administered at a dose which is above 0 to 75%, such as 20-75% (e.g. 25%, 30%, 35%, 40%, 45%, 50%, 55%, 60%, 65%, or 70%), or such as 25-50%, of the therapeutically effective dose.

The reduction of the dose of the 1-aminoalkylcyclohexane derivative, such as neramexane or a pharmaceutically acceptable salt thereof, such as neramexane mesylate, between the administration of the therapeutically effective dose and the administration of 0-75%, such as above 0-75%, or 20-75%, such as 25-50%, of the therapeutically effective dose may be performed stepwise. The dose of 50 mg neramexane mesylate daily may, for example, be reduced to a dose of 25 mg by 12.5 mg steps, wherein the 37.5 mg dose is, for example, administered for at least one week. The dose of 75 mg neramexane mesylate daily may, for example, be reduced to a dose of 25 mg/day. This may be performed by 12.5 mg steps, wherein the 62.5 mg/day, 50 mg/day, and 37.5 mg/day doses may, for example, be administered for at least one week, respectively. Alternatively, the dose of 75 mg neramexane mesylate daily may, for example, be reduced to a dose of 50 mg/day. This may be performed by 12.5 mg steps, wherein the 62.5 mg/day dose may, for example, be administered for at least one week. In a further embodiment, the dose of 75 mg neramexane mesylate daily may, for example, be reduced to a dose of 12.5 mg/day. This may be performed by 12.5 mg steps, wherein the 62.5 mg/day dose, the 50 mg/day dose, the 37.5 mg/day dose, and the 25 mg/day dose may, respectively, for example, be administered for at least one week.

A further aspect of the invention relates to a treatment regimen for an individual afflicted with nystagmus, comprising administering to the individual a therapeutically effective amount of a 1-aminoalkylcyclohexane derivative, such as neramexane or a pharmaceutically acceptable salt thereof, such as neramexane mesylate, wherein the therapeutically effective amount of the 1-aminoalkylcyclohexane derivative is administered daily for a period of at least three (3) months, followed by a period of at least one month wherein the 1-aminoalkylcyclohexane derivative is administered at a dose which is 0-75%, such as above 0-75%, or 20-75% or 25-50% of the therapeutically effective dose, with the treatment regimen being repeated after a recurrence of nystagmus.

In one embodiment, patients who have had a nystagmus relapse continue on the therapeutically effective dose for at least three months, such as for at least one year, before the dose is reduced to a maintenance dose.

Alternatively, the treatment regimen may be repeated after a specific period, such as after administering at a dose which is 0-75%, or above 0-75%, or 20-75%, such as 25-50% of the therapeutically effective dose for a period of from three (3) to six (6) months (e.g. 3, 4, 5, or 6 months).

In a further aspect of the invention, the therapeutically effective amount of the 1-aminoalkylcyclohexane derivative such as a neramexane or a pharmaceutically acceptable salt thereof, such as neramexane mesylate, is administered daily for a first period of at least three (3) months, followed by a second period of at least one month, such as at least three (3) months, wherein the 1-aminoalkylcyclohexane derivative is not administered, with the treatment regimen being repeated after said (second) period. Said order of periods with and without treatment can be repeated several times, usually depending upon the progress of the condition within the subject treated.

The treatment regimen may be repeated after a specific period, such as after a period of from three (3) to six (6) months (e.g. 3, 4, 5, or 6 months).

According to the invention, the dose reduction, as well as the dose increase, may be performed stepwise.

In a further aspect of the invention a therapeutically effective amount of neramexane or a pharmaceutically acceptable salt thereof, such as neramexane mesylate, is administered to the subject, wherein the therapeutically effective amount of said compound is administered daily for a period of at least three (3) months, followed by a period of at least one (1) month wherein said compound is administered at a dose which is 0-75% or above 0-75%, such as 20-75%, of the therapeutically effective dose, with the treatment being repeated as necessary.

In a further aspect of the invention neramexane mesylate is administered in a range from about 5 mg to about 150 mg/day during the first period, or in a range from about 5 mg to about 100 mg/day during the first period, or in a range from about 5 mg to about 75 mg/day, during the first period, or at about 50 mg/day during the first period, or at about 75 mg/day during the first period.

In a further aspect of the invention the 1-aminoalkylcyclohexane derivative such as neramexane or a pharmaceutically acceptable salt thereof, such as neramexane mesylate, is administered once a day, twice a day (b.i.d.), or three times a day. The administration may be achieved via an immediate release formulation, or a modified release formulation.

A further aspect of the invention relates to the derivative/use specified above, wherein the 1-aminoalkylcyclohexane derivative such as neramexane or a pharmaceutically acceptable salt thereof, such as neramexane mesylate, is administered in a titration scheme which provides quick and safe attainment of an effective dose.

A further aspect of the invention relates to the derivative/use specified above wherein said titration scheme comprises up-titration of a 1-aminoalkylcyclohexane derivative, such as neramexane or a pharmaceutically acceptable salt thereof, such as neramexane mesylate, over a period of from four to five weeks to achieve an effective dose.

In a further aspect of the invention the titration scheme comprises up-titration of a 1-aminoalkylcyclohexane derivative, such as neramexane or a pharmaceutically acceptable salt thereof, such as neramexane mesylate, over a period of from four to five weeks to achieve an effective dose of from 5 to 150 mg per day.

In a further aspect of the invention, the titration scheme comprises up-titration of a 1-aminoalkylcyclohexane derivative, such as neramexane or a pharmaceutically acceptable salt thereof, such as neramexane mesylate, over a period of from four to five weeks to achieve an effective dose of from 50 to 75 mg per day.

In a further aspect of the invention, the titration scheme comprises up-titration of a 1-aminoalkylcyclohexane derivative, such as neramexane or a pharmaceutically acceptable salt thereof, such as neramexane mesylate, in increasing dosages of 25 mg or 12.5 mg steps at weekly intervals.

In a further aspect of the invention, the 1-aminoalkylcyclohexane derivative is neramexane or a pharmaceutically acceptable salt thereof, such as neramexane mesylate.

In a further aspect of the invention, the titration scheme comprises up-titration of neramexane or a pharmaceutically acceptable salt thereof, such as neramexane mesylate, over a period of four weeks to achieve an effective dose of 50 mg per day while minimizing side effects.

In a further aspect of the invention, neramexane or a pharmaceutically acceptable salt thereof, such as neramexane mesylate, is administered according to the following schedule: once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week.

In a further aspect of the invention, neramexane or a pharmaceutically acceptable salt thereof, such as neramexane mesylate, is administered according to the following schedule: once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week, wherein, in weeks during which mixed doses are administered, the dose comprising the higher concentration is administered in the second daily dose.

In a further aspect of the invention, the titration scheme comprises up-titration of neramexane or a pharmaceutically acceptable salt thereof, such as neramexane mesylate, which titration scheme allows for up-titration of neramexane or a pharmaceutically acceptable salt thereof, such as neramexane mesylate, over a period of five weeks to achieve an effective dose of 75 mg per day while minimizing side effects.

In a further aspect of the invention, neramexane or a pharmaceutically acceptable salt thereof, such as neramexane mesylate, is administered according to the following schedule: once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week, and twice daily, wherein each dose is 37.5 mg for the fifth week, wherein, in weeks during which mixed doses are administered, the dose comprising the higher concentration is administered in the second daily dose.

In a further aspect of the invention, neramexane or a pharmaceutically acceptable salt thereof, such as neramexane mesylate, is administered in the form of a modified release once daily formulation according to the following schedule: once daily at a dose of 12.5 mg for the first week, once daily at a dose of 25 mg for the second week, once daily at a dose of 37.5 mg for the third week, once daily at a dose of 50 mg for the fourth week, for subjects with a weight up to 90 kg, and—in addition to the above—once daily a dose of 75 mg for subjects having a weight of more than 90 kg.

In a further aspect of the invention, the composition comprising neramexane or a pharmaceutically acceptable salt thereof, such as neramexane mesylate, is administered in the form of a modified release once daily formulation according to the following schedule: once daily at a dose of 25 mg for the first week, once daily at a dose of 50 mg for the second week, for subjects with a weight up to 90 kg, and—in addition to the above—once daily a dose of 75 mg for subjects having a weight of more than 90 kg.

In a further aspect, the invention relates to at least one of the above specified titration schemes, wherein a composition comprising neramexane mesylate is administered according to such schedule. If another pharmaceutically acceptable salt, a solvate, an isomer, a conjugate, a prodrug or a derivative thereof, such as neramexane hydrochloride, is administered, according to a respective titration scheme, equimolar amounts of another pharmaceutically acceptable salt, a solvate, an isomer, a conjugate, a prodrug or a derivative thereof, such as neramexane hydrochloride, may also be suitable.

A further aspect of the invention relates to the treatment of an individual diagnosed with nystagmus, comprising administering to the individual an effective amount of neramexane, or a prodrug, derivative, or pharmaceutically acceptable salt thereof.

A further aspect of the invention relates to the treatment of an individual diagnosed with nystagmus, comprising administering neramexane or a pharmaceutically acceptable salt thereof, such as neramexane mesylate, to the individual in a range from about 5 mg to about 150 mg/day, in a range from about 5 mg to about 100 mg/day, in a range from about 5 mg to about 75 mg/day, at about 50 mg/day or at about 75 mg/day.

A further aspect of the invention relates to the use of neramexane or a pharmaceutically acceptable salt thereof, such as neramexane mesylate, in the manufacture of a medicament for the treatment of nystagmus, wherein the medicament is manufactured for administration at a dose from about 5 mg to about 150 mg/day, at a dose in a range from about 5 mg to about 100 mg/day, at a dose in a range from about 5 mg to about 75 mg/day, at a dose about 50 mg/day or at a dose about 75 mg/day.

A further aspect of the invention relates to the use of neramexane or a pharmaceutically acceptable salt thereof, such as neramexane mesylate, in the manufacture of a medicament for treatment of nystagmus.

A further aspect of the invention relates to a pharmaceutical composition for the treatment of nystagmus comprising a therapeutically effective amount of neramexane or a pharmaceutically acceptable salt thereof, such as neramexane mesylate, and at least one pharmaceutically acceptable carrier or excipient.

A further aspect of the invention relates to a pharmaceutical composition for the treatment of nystagmus comprising a therapeutically effective amount of neramexane or a pharmaceutically acceptable salt thereof, such as neramexane mesylate, in an immediate or modified release formulation.

A further aspect of the invention relates to the use of neramexane or a pharmaceutically acceptable salt thereof, such as neramexane mesylate, in the manufacture of a medicament for the treatment of nystagmus, wherein the medicament is manufactured to provide neramexane or a pharmaceutically acceptable salt thereof, such as neramexane mesylate, in an immediate or modified release formulation.

A further aspect of the invention relates to the treatment of an individual diagnosed with nystagmus comprising administering to the individual neramexane or a pharmaceutically acceptable salt thereof, such as neramexane mesylate, and an additional pharmaceutical agent which has been shown to be effective in treating nystagmus.

A further aspect of the invention relates to the treatment of an individual diagnosed with nystagmus comprising administering to the individual neramexane or a pharmaceutically acceptable salt thereof, such as neramexane mesylate, and an additional pharmaceutical agent which has been shown to be effective in treating nystagmus, wherein neramexane or a pharmaceutically acceptable salt thereof, such as neramexane mesylate, and the additional pharmaceutical agent are administered conjointly or in a single formulation.

A further aspect of the invention relates to the use of neramexane or a pharmaceutically acceptable salt thereof, such as neramexane mesylate, in combination with an additional pharmaceutical agent which has been shown to be effective in treating nystagmus, in the manufacture of a medicament for the treatment of nystagmus.

A further aspect of the invention relates to the treatment of individuals diagnosed with nystagmus comprising administering to the individual neramexane or a pharmaceutically acceptable salt thereof, such as neramexane mesylate, and a pharmaceutical agent selected from memantine, gabapentin, vigabatrin, pregabalin, 4-aminopyridine, 3,4-diaminopyridine, baclofen, scopolamine, and clonazepam.

A further aspect of the invention relates to the use of neramexane or a pharmaceutically acceptable salt thereof, such as neramexane mesylate, in combination with an additional pharmaceutical agent which is selected from memantine, gabapentin, vigabatrin, pregabalin, 4-aminopyridine, 3,4-diaminopyridine, baclofen, scopolamine, and clonazepam in the manufacture of a medicament for the treatment of nystagmus.

A further aspect of the invention relates to a pharmaceutical composition comprising a therapeutically effective amount neramexane or a pharmaceutically acceptable salt thereof, such as neramexane mesylate, in combination with other therapies for nystagmus and, optionally, at least one pharmaceutically acceptable carrier or excipient.

A further aspect of the invention includes a pharmaceutical composition comprising a therapeutically effective amount neramexane or a pharmaceutically acceptable salt thereof, such as neramexane mesylate, a pharmaceutical agent selected from memantine, gabapentin, baclofen, vigabatrin, pregabalin, 4-aminopyridine, 3,4-diaminopyridine, scopolamine, and clonazepam, and at least one pharmaceutically acceptable carrier or excipient.

A further aspect of the invention includes a pharmaceutical composition comprising a therapeutically effective amount of neramexane or a pharmaceutically acceptable salt thereof, such as neramexane mesylate, and a pharmaceutical agent selected from memantine, gabapentin, vigabatrin, pregabalin, 4-aminopyridine, 3,4-diaminopyridine, baclofen, scopolamine and clonazepam, in an immediate or modified release formulation.

A further aspect of the invention relates to the use of neramexane or a pharmaceutically acceptable salt thereof, such as neramexane mesylate, in the manufacture of a medicament for the treatment of nystagmus, wherein the medicament is manufactured for administration of neramexane or a pharmaceutically acceptable salt thereof, such as neramexane mesylate, once a day, twice a day (b.i.d.), or three times a day.

The active ingredient (the 1-aminoalkylcyclohexane derivative, such as neramexane or a pharmaceutically acceptable salt thereof, such as neramexane mesylate) or the composition of the present invention may be used for the treatment of nystagmus, wherein the medicament is adapted to or appropriately prepared for a specific administration as disclosed herein (e.g., intervallic treatment, maintenance therapy, once-a-day, twice-a-day administration, or three times a day administration). For this purpose the package leaflet and/or the patient information contains corresponding information.

### DETAILED DESCRIPTION OF THE INVENTION

The term “treat” is used herein to mean to relieve or alleviate at least one symptom of a disease or condition in a subject. Within the meaning of the present invention, the term “treat” also denotes to arrest, delay the onset (i.e., the period prior to clinical manifestation of a disease) and/or reduce the risk of developing or worsening a disease.

As used herein, the term “nystagmus” encompasses congenital and acquired forms of the disease, including subtypes thereof. Further the term nystagmus encompasses pathological forms of the disease and nystagmus resulting from toxic or metabolic causes, including subtypes thereof. The term nystagmus also includes ocular tremor or oscillopsia. Moreover, nystagmus also encompasses downbeat nystagmus, upbeat nystagmus, seesaw nystagmus, periodic alternating nystagmus, and acquired pendular nystagmus. Conditions/diseases to be mentioned as falling under the category “congenital nystagmus” include, but are not limited to, idiopathy, albinism, aniridia, Leber's congenital amaurosis, bilateral optic nerve hypoplasia, bilateral congenital cataracts, rod monochromatism, optic nerve or macular disease, persistent tunica vasculosa lentis, latent nystagmus and nystagmus blockage syndrome. Examples of diseases/conditions falling under the definition of “pathological nystagmus” include, but are not limited to, peripheral nystagmus, positional nystagmus, gaze induced nystagmus, post head shake nystagmus, spontaneous nystagmus as well as central nystagmus. Conditions/disorders falling under the definition “acquired nystagmus” include, but are not limited to benign paroxysmal positional vertigo, head trauma, stroke, Ménière's disease and other balance disorders, multiple sclerosis, brain tumors, Wernicke-Korsakoff syndrome, encephalopathy, lateral medullary syndrome, optic nerve hypoplasia, Nooan syndrome, Pelizaeus-Merzbacher disease, superior canal dehiscence syndrome, tullio phenomenon, Horner's syndrome. Conditions/disorders falling under the definition “Nystagmus resulting from toxic or metabolic causes” include, but are not limited to intoxications with alcohol, lithium, barbiturates, phenyloin, salicylates, benzodiazepines, LSD, phenylcyclidine, aminoglycosides, anticonvulsants, sedatives, methylenedioxymethamphetamine, Wernicke's encephalopathy, thiamine deficiency.

Furthermore, 1-aminoalkylcyclohexane derivatives, such as neramexane or a pharmaceutically acceptable salt thereof, such as neramexane mesylate, may be also used for the treatment of ocular disorders/diseases in general. Such ocular diseases include, but are not limited to ocular hypertension, glaucoma, low-tension glaucome, diabetic retinopathy, age-related macular degeneration, diabetic macular edema, ischemic optic neuropathy, optic nerve trauma, optic neuritis, retinal vein occlusion, retinal artery occlusion, retinal edema, retinal ischemia, damages of the retina caused by e.g. photocoagulation and accidental laser injuries.

Furthermore, said 1-aminoalkylcyclohexane derivatives, such as neramexane or a pharmaceutically acceptable salt thereof, such as neramexane mesylate, may be also used for the treatment of superior oblique myokymia.

The term 1-aminoalkylcyclohexane derivative is used herein to describe a 1-aminoalkylcyclohexane or a compound derived from 1-aminoalkylcyclohexane, e.g., pharmaceutically acceptable salts of 1-aminoalkylcyclohexanes.

The 1-aminoalkylcyclohexane derivatives of the present invention may be represented by the general formula (I):

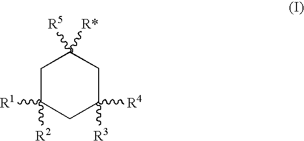

wherein R* is —(CH₂)ₙ—(CR⁸R⁷)ₘ—NR⁸R⁹
wherein n+m=0, 1, or 2
wherein R¹ through R⁷ are independently selected from the group consisting of hydrogen and C₁₋₆alkyl, wherein R⁸ and R⁹ are independently selected from the group consisting of hydrogen and C₁₋₆alkyl or together represent lower-alkylene —(CH₂)ₓ— wherein x is 2 to 5, inclusive, and optical isomers, enantiomers, hydrates, and pharmaceutically-acceptable salts thereof.

Non-limiting examples of the 1-aminoalkylcyclohexanes used according to the present invention include:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1 amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5,5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl-1)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,5S)trans-5-ethyl-1,3,3-trimethylcyclohexane,
1-amino-(1S,5S)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,5S)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,5S)trans-5-ethyl-1,3,3-trimethylcyclohexyl]pyrrolidine or piperidine,
N-(1-ethyl-3,3,5,5-tetramethylcyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
   and optical isomers, diastereomers, enantiomers, hydrates, their pharmaceutically acceptable salts, and mixtures thereof.

Neramexane (1-amino-1,3,3,5,5-pentamethylcyclohexane) is disclosed in U.S. Pat. Nos. 6,034,134 and 6,071,966. Neramexane, may be used according to the invention in the form of any of its pharmaceutically acceptable salts, solvates, isomers, conjugates, prodrugs and derivatives, any references to neramexane in this description should be understood as also referring to such salts, solvates, isomers, conjugates, prodrugs and derivatives.

Pharmaceutically acceptable salts of neramexane include, but are not limited to, acid addition salts, such as those made with hydrochloric, methylsulfonic, hydrobromic, hydroiodic, perchloric, sulfuric, nitric, phosphoric, acetic, propionic, glycolic, lactic, pyruvic, malonic, succinic, fumaric, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. All of these salts (or other similar salts) may be prepared by conventional means. The nature of the salt is not critical, provided that it is non-toxic and does not substantially interfere with the desired pharmacological activity.

The term “analog” or “derivative” is used herein in the conventional pharmaceutical sense, to refer to a molecule that structurally resembles a reference molecule (such as neramexane), but has been modified in a targeted and controlled manner to replace one or more specific substituents of the referent molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

The term “therapeutically effective” applied to a dose or an amount refers to that quantity of a compound or pharmaceutical composition that is sufficient to result in a desired activity upon administration to a mammal in need thereof.

The term “pharmaceutically acceptable”, as used in connection with compositions of the invention, refers to molecular entities and other ingredients of such compositions that are physiologically tolerable and do not typically produce untoward reactions when administered to a mammal (e.g. a human). As used herein, the term “pharmaceutically acceptable” also or alternatively means approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound (e.g., neramexane) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A. R. Gennaro, 20^{th} Edition.

The term “about” or “approximately” usually means within 20%, alternatively within 10%, including within 5% of a given value or range. Alternatively, especially in biological systems, the term “about” means within about a log (i.e., an order of magnitude), including within a factor of two of a given value.

The term “titration scheme” is meant to be a method of treatment as discussed herein, wherein patients are treated for a disease or a condition wherein at least two different dosages (doses) of one or more 1-aminoalkylcyclohexane derivatives, e.g. in the form of a pharmaceutical compositions useful in treating such condition are administered in a step-wise manner in a once daily or multiple times per day manner and wherein lower doses are administered earlier in the treatment and higher doses are administered during subsequent treatment weeks. Optionally, in those treatment weeks wherein different dosages are administered on the same day, the titration scheme may provide for the administration of a lower dosage in the morning and a higher dosage in the evening, thereby minimizing drug-induced side effects during the most productive hours of the day.

In conjunction with the methods of the present invention, also provided are pharmaceutical compositions comprising a therapeutically effective amount of a 1-aminoalkylcyclohexdane derivative such as neramexane or a pharmaceutically acceptable salt thereof, such as neramexane mesylate. The compositions of the invention may further comprise a carrier or excipient (all pharmaceutically acceptable). The compositions may be formulated for once-a-day administration, twice-a-day administration, or three times a day administration.

According to the present invention, the dosage form of the 1-aminoalkylcyclohexane derivative, such as neramexane may be a solid, semisolid, or liquid formulation according to the following.

The 1-aminoalkylcyclohexane derivative, such as neramexane may be administered orally, topically, parenterally, or mucosally (e.g., buccally, by inhalation, or rectally) in dosage unit formulations containing conventional non-toxic pharmaceutically acceptable carriers. In another embodiment for administration to pediatric subjects, the 1-aminoalkylcyclohexane derivative, such as neramexane may be formulated as a flavored liquid (e.g., peppermint flavor). The 1-aminoalkylcyclohexane derivative, such as neramexane or a pharmaceutically acceptable salt thereof, such as neramexane mesylate may be administered orally in the form of a capsule, a tablet, or the like, or as a semi-solid, or liquid formulation (see Remington's Pharmaceutical Sciences, 20^{th} Edition, by A. R. Gennaro).

For oral administration in the form of a tablet or capsule, the 1-aminoalkylcyclohexane derivative, such as neramexane or a pharmaceutically acceptable salt thereof, such as neramexane mesylate, may be combined with non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets may be coated with a concentrated sugar solution which may contain e.g., gum arabic, gelatine, talcum, titanium dioxide, and the like. Alternatively, the tablets can be coated with a polymer that dissolves in a readily volatile organic solvent or mixture of organic solvents. In specific embodiments, neramexane is formulated in to immediate-release (IR) or modified-release (MR) tablets. Immediate release solid dosage forms permit the release of most or all of the active ingredient over a short period of time, such as 60 minutes or less, and make rapid absorption of the drug possible (immediate release formulations of neramexane are disclosed in US Published Application Nos. 2006/0002999 and 2006/0198884, the subject matter of which is hereby incorporated by reference). Modified release solid oral dosage forms permit the sustained release of the active ingredient over an extended period of time in an effort to maintain therapeutically effective plasma levels over similarly extended time intervals and/or to modify other pharmacokinetic properties of the active ingredient (modified release formulations of neramexane are disclosed in U.S. application Ser. No. 11/604,986, the subject matter of which is hereby incorporated by reference).

For the formulation of soft gelatin capsules, the active substances may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

The compositions of the invention can also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers may be used in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

Formulation of the 1-aminoalkylcyclohexane derivative, such as neramexane or a pharmaceutically acceptable salt thereof, such as neramexane mesylate, in a semi-solid or liquid form may also be used. the 1-aminoalkylcyclohexane derivative, such as neramexane or a pharmaceutically acceptable salt thereof, such as neramexane mesylate, may constitute between 0.1 and 99% by weight of the formulation, more specifically between 0.5 and 20% by weight for formulations intended for injection and between 0.2 and 50% by weight for formulations suitable for oral administration.

In one embodiment of the invention, the 1-aminoalkylcyclohexane derivative, such as neramexane or a pharmaceutically acceptable salt thereof, such as neramexane mesylate, is administered in a modified release formulation. Modified release dosage forms provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, thereby eliminating or reducing sharp peaks.

A modified release form dosage may comprise a core either coated with or containing a drug. The core being is then coated with a release modifying polymer within which the drug is dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In another embodiment of the invention, the 1-aminoalkylcyclohexane derivative, such as neramexane or a pharmaceutically acceptable salt thereof, such as neramexane mesylate, is formulated in an oral, liquid formulation. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. Oral liquid formulations of neramexane are described in PCT International Application No. PCT/US2004/037026, the subject matter of which is hereby incorporated by reference.

For oral administration in liquid form, the 1-aminoalkylcyclohexane derivative, such as neramexane or a pharmaceutically acceptable salt thereof, such as neramexane mesylate, may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of neramexane, with the balance being sugar and mixture of ethanol, water, glycerol and propylene glycol. Optionally, such liquid formulations may contain coloring agents, flavoring agents, saccharine and carboxymethyl-cellulose as a thickening agent or other excipients.

In another embodiment, a therapeutically effective amount of the 1-aminoalkylcyclohexane derivative, such as neramexane or a pharmaceutically acceptable salt thereof, such as neramexane mesylate, is administered in an oral solution containing a preservative, a sweetener, a solubilizer, and a solvent. The oral solution may include one or more buffers, flavorings, or additional excipients. In a further embodiment, a peppermint or other flavoring is added to the neramexane derivative oral liquid formulation.

For administration by inhalation, the 1-aminoalkylcyclohexane derivative, such as neramexane or a pharmaceutically acceptable salt thereof, such as neramexane mesylate, may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Solutions for parenteral applications by injection may be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules.

Dosage units for rectal application may be solutions or suspensions or may be prepared in the form of suppositories or retention enemas comprising neramexane in a mixture with a neutral fatty base, or gelatin rectal capsules comprising the active substances in admixture with vegetable oil or paraffin oil.

The formulations of the invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. Alternatively, the active ingredient may be in powder form for reconstitution with a suitable vehicle, e.g., sterile pyrogen-free water, before use.

The invention also provides a pharmaceutical pack or kit comprising one or more containers containing the 1-aminoalkylcyclohexane derivative, such as neramexane or a pharmaceutically acceptable salt thereof, such as neramexane mesylate, and, optionally, more of the ingredients of the formulation. In a specific embodiment, neramexane is provided as an oral solution (2 mg/ml) for administration with the use of a 2 teaspoon capacity syringe (dosage KORC®). Each oral syringe has blue hatch marks for measurement, with lines on the right side of the syringe (tip down) representing tsp units, and those on the left representing ml units.

The optimal therapeutically effective amount may be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge.

Dosage units for rectal application may be solutions or suspensions or may be prepared in the form of suppositories or retention enemas comprising neramexane or a pharmaceutically acceptable salt thereof, such as neramexane mesylate, in a mixture with a neutral fatty base, or gelatin rectal capsules comprising the active substances in admixture with vegetable oil or paraffin oil.

Toxicity and therapeutic efficacy of the compositions of the invention may be determined by standard pharmaceutical procedures in experimental animals, e.g., by determining the LD50 (the dose lethal to 50% of the population) and the ED50 (the dose therapeutically effective in 50% of the population). The dose ratio between therapeutic and toxic effects is the therapeutic index and it may be expressed as the ratio LD50/ED50. Compositions that exhibit large therapeutic indices are preferred.

Suitable daily doses of the active compounds of the invention in therapeutic treatment of humans are about 0.01-10 mg/kg bodyweight on peroral administration and 0.001-10 mg/kg bodyweight on parenteral administration. For adults, suitable daily doses of neramexane are within the range from about 5 mg to about 150 mg per day, such as from about 5 mg to about 120 mg, from about 5 mg to about 100 mg, or from about 5 mg to about 50 mg per day. For pediatric subjects aged 4-14, neramexane may be administered as an oral, liquid dosage form, at about 0.5 mg/day, up to a maximum dose of 10 mg/day.

Suitable daily doses of the active compounds of the invention in therapeutic treatment of humans are about 0.01-10 mg/kg bodyweight on peroral administration and 0.001-10 mg/kg bodyweight on parenteral administration. For example, for adults, suitable daily doses of neramexane mesylate are within the range from about 5 mg to about 150 mg per day, such as from about 5 mg to about 120 mg, from about 5 mg to about 100 mg, or from about 5 mg to about 75 mg, or from about 5 mg to about 50 mg, such as 25 mg or 50 mg, per day. As a therapeutically effective amount of the present invention a daily dose of neramexane mesylate may be administered within the range from about 20 mg to 150 mg, such as from 25 mg to 100 mg (e.g. 30 mg, 35 mg, 40 mg, 45 mg, 50 mg, 55 mg, 60 mg, 65 mg, 70 mg, 75 mg, 80 mg, 85 mg, 90 mg, or 95 mg), such as from 50 mg to 75 mg. An equimolar amount of another pharmaceutically acceptable salt, a solvate, an isomer, a conjugate, a prodrug or a derivative thereof, such as neramexane hydrochloride, is also suitable. For pediatric subjects aged 4-14, neramexane (e.g. neramexane mesylate) may be administered as an oral, liquid dosage form, at about 0.5 mg/day, up to a maximum dose of 10 mg/day.

Treatment duration may be short-term, e.g., several weeks (for example 8-14 weeks), or long-term until the attending physician deems that further administration is no longer is necessary.

The 1-aminoalkylcyclohexane derivative, such as neramexane or a pharmaceutically acceptable salt thereof, such as neramexane mesylate, may be administered as a monotherapy, or in combination with another agent prescribed for the treatment of nystagmus.

The term “combination” applied to active ingredients is used herein to define a single pharmaceutical composition (formulation) comprising two active agents (e.g., a pharmaceutical composition comprising a 1-aminoalkylcyclohexane derivative, such as neramexane or a pharmaceutically acceptable salt thereof, such as neramexane mesylate, and another agent prescribed for the treatment of nystagmus) or two separate pharmaceutical compositions, each comprising an active agent (e.g. a pharmaceutical composition comprising a neramexane or another agent prescribed for the treatment of nystagmus), to be administered conjointly.

Within the meaning of the present invention, the term “conjoint administration” is used to refer to administration of a 1-aminoalkylcyclohexane derivative, such as neramexane or a pharmaceutically acceptable salt thereof, such as neramexane mesylate, and a second active agent (e.g. another agent prescribed for the treatment of nystagmus) simultaneously in one composition, or simultaneously in different compositions, or sequentially. For the sequential administration to be considered “conjoint”, however, the 1-aminoalkylcyclohexane derivative, such as neramexane or a pharmaceutically acceptable salt thereof, such as neramexane mesylate, and the second active agent must be administered separated by a time interval which still permits the resultant beneficial effect for treating nystagmus in a mammal.

### EXAMPLES

The following example illustrates the invention without limiting its scope.

### Example 1

### Double Blind Placebo Controlled Pilot Trial of Neramexane for Treatment of Nystagmus

The objective of this pilot project is to conduct a clinical trial to assess the efficacy of neramexane as a treatment for nystagmus. Nystagmus patients treated with neramexane may be expected to demonstrate an improvement in primary (e.g., visual acuity) and secondary (e.g., nystagmus intensity and expanded nystagmus acuity function [NAFX], reading ability etc.) outcomes as compared to placebo treated patients.

### Study Design

The primary objective of this study is to investigate the safety and efficacy of Neramexane mesylate at daily doses of up to 75 mg in the treatment of congenital, idiopathic nystagmus in comparison to placebo. In addition, a subgroup of up to 20 MS patients suffering from acquired nystagmus are included and analyzed in an exploratory manner. In both patient groups, effects of treatment is assessed in comparison with placebo using a two-period cross-over design (two treatment periods of 7 weeks each, separated by a wash-out phase.

The study is designed as a randomized, double-blind placebo-controlled, pilot study using a two-period cross-over design. Suitable patients are randomly assigned to one of the two treatment sequences of the cross-over trial (sequence A-B or B-A), i.e. double-blind treatment with Neramexane (A) and matching placebo tablets (B).

### Statistical Procedures and Populations for Analysis

In order to be eligible to participate in the study, patients must meet the following criteria:

Patients with congenital nystagmus (no pathology other than nystagmus; primary analysis population) or acquired nystagmus subsequent to MS (exploratory analysis only)
male or female outpatients aged between 18 and 80 years (inclusive) at screening;
patients with a metric visual acuity (VA) of 6/9 or lower 6/9);
for females of childbearing potential (last menses less than one year prior to enrollment): negative pregnancy test at baseline; either surgically sterile or use of a medically accepted method of contraception during the entire duration of the study;
congenital idiopathic nystagmus patients: uneventful gross neurological examination;
MS patients: diagnosis ‘multiple sclerosis’ as confirmed on the basis of McDonald criteria;
normal results of electroretinography (ERG) and visually evoked potential (VEP) testing; historical results may be accepted (period of acceptability to be defined)
patients having given written informed consent;
patient expected and willing to comply with the scheduled visits, dosing scheme (in particular placebo), and other requirements of the study;
results of normal physical examination, electrocardiogram (ECG), and safety laboratory at screening, or abnormal findings which are judged not clinically significant by the investigator;
Refraction prior to enrolment and, if required, prescription and receipt of new glasses or contact lenses prior to baseline visual acuity measurement

Patients meeting any of the following criteria are excluded from the study:
multiple sclerosis patients: history of epileptic seizures or a history of cardiac arrhythmias (as evidenced by ECG); patients presenting with acute relapse;
patients presenting with acute optic neuritis;
diseases affecting the vestibular organ in the inner ear;
patients with evidence of neurologic disorders other than congenital idiopathic nystagmus and/or acquired nystagmus (secondary to MS), including, but not limited to epilepsy, infranuclear disorders such as benign paroxysmal positional vertigo (BBPV), vestibular neuritis, Meniere's disease, superior canal dehiscence syndrome, vestibular paroxysmia, or superior oblique myokymia;
nystagmus due to tumour lesions (e.g. pituitary tumours);
nystagmus due inflammatory processes other than MS;
patients with known hypersensitivity or intolerance to Neramexane, amantadine, or memantine;
patients with intake of non-permitted concomitant medication (e.g., sedatives; anticonvulsants; drugs interfering with the GABA-ergic system, including baclofen, clonazepam, gabapentin, pregabalin, and vigabatrin; NMDA antagonists, including amantadine and memantine; scopolamine; potassium channel blocking agents, including 4-aminopyridine and 3,4-diaminopyridine; sodium channel blocking agents, including lamotrigine);
prior exposure to gabapentin or memantine;
simultaneous participation in another clinical trial;
patients having participated in a clinical study and/or having received an investigational compound within two months prior to screening;
patients with evidence of clinically significant and active pulmonary, gastrointestinal, renal, hepatic, endocrine, or cardiovascular system disease (patients with medically controlled stable hypertension and/or diabetes may be enrolled);
patients with an oncology diagnosis (hematology or solid tumor) currently undergoing treatment, completion of treatment within the past six months, or who still have evidence of active disease;
known or suspected alcoholism or drug abuse;
employees or direct relatives of an employee of the investigational site or medical students of the University (waiver may be obtained);
any other condition which in the opinion of the investigator precludes enrollment of the patient.

The scheduled visits for evaluation of each patient are as follows:

Visit 1 (initial screening): After signing the consent form, the subject undergoes a gross neurological and ophthamological evaluation. Visual acuity as well as secondary parameters, including nystagmus intensity, and reading speed are evaluated. Patient eligibility for study is evaluated via review of inclusion/exclusion criteria.

Visit 2 (baseline for first seven week sequence): Subject is evaluated for study eligibility based on a review of the inclusion/exclusion criteria. Study procedures as well as concomitant medications are reviewed with the subject. Visual acuity as well as secondary parameters, including nystagmus intensity and reading speed are evaluated. Subject is enrolled in the study and medication is dispensed as described below.

Visit 3: This visit occurs at the end of the first 3-week up-tritration sequence. Review of concomitant medications as well as the occurrence of adverse events since the last visit is conducted with subject. Visual acuity as well as secondary parameters, including nystagmus intensity and reading speed are evaluated. Medication is dispensed as described below.

Visit 4: This visit occurs at the end of the first 4-week constant-dose double-blind sequence. Review of concomitant medications as well as the occurrence of adverse events since the last visit is conducted with subject. Visual acuity as well as secondary parameters, including nystagmus intensity and reading speed are evaluated.

Visit 5 (baseline for second seven week sequence): Following a four week wash out period, study procedures as well as concomitant medications are reviewed with the subject. Visual acuity as well as secondary parameters, including nystagmus intensity and reading speed are evaluated. Medication is dispensed as described below.

Visit 6: This visit occurs at the end of the second 3-week up-tritration sequence. Review of concomitant medications as well as the occurrence of adverse events since the last visit is conducted with subject. Visual acuity as well as secondary parameters, including nystagmus intensity and reading speed are evaluated. Medication is dispensed as described below.

Visit 7: This visit occurs at the end of the second 4-week constant-dose double-blind sequence. Review of concomitant medications as well as the occurrence of adverse events since the last visit is conducted with subject. Visual acuity as well as secondary parameters, including nystagmus intensity and reading speed are evaluated.

Visit 8: This visit occurs thirty days after the last dose. Visual acuity as well as secondary parameters, including nystagmus intensity and reading speed are evaluated.

### Administration of Neramexane

Neramexane mesylate 25 mg modified release tablets and matching placebo tablets are administered as film coated tablets.

Neramexane (or placebo) is uptitrated to a maximum daily dose of 75 mg, starting with a daily dose of 25 mg for one week, and increasing dosage in 25 mg steps at weekly intervals.

Treatment is started on an outpatient basis in the morning of study Day 1. The daily starting dose is 25 mg Neramexane to be taken for 7 days (1 tablet daily in the morning for one week). Thereafter, the daily Neramexane dose is increased to 50 mg for another 7 days (two tablets in the morning for one week). Patients who experience no dose-limiting adverse effects of treatment by the end of week 2 are uptitrated to 75 mg Neramexane per day for up to 28 days (three tablets in the morning for 4 weeks). Patients who do not tolerate a dose level receive a reduction in dosage to the last well tolerated dose for the remainder of the total scheduled treatment duration of 7 weeks. For example, patients who do not tolerate a 75 mg dose are allowed to step back to a 50 mg dose. Patients are then asked to stay on the 50 mg dose for the remainder of the total scheduled treatment duration of 7 weeks. This dosing regimen is shown in Table 1.

TABLE 1
Administration of Neramexane

| | | | 4 weeks double-blind |
|---|---|---|---|
| 3 weeks uptitration | | | period |
| Week 1 | Week 2 | Week 3 | Weeks 4 to 7 |
| 25 mg/d | 50 mg/d | 75 mg/d | 75 mg/d |

### Efficacy

Visual acuity (VA) is the standard by which the outcomes of drug therapy in nystagmus are measured in the framework of clinical trials. The distance visual acuity measurement has been used extensively to evaluate vision because it is relatively simple and objective. In this study, the logarithm of the minimum angle of resolution (LogMAR) is used as a primary response variable

### Primary Outcome

Change from baseline in best corrected visual acuity after 7 weeks of treatment

### Secondary Outcomes

Change from baseline in visual acuity at increasing gaze eccentricity
Number/frequency of responder
Change from baseline in Nystagmus intensity
Change from baseline in Expanded Nystagmus Acuity Function (NAFX)
Change from baseline in reading speed
Investigator Assessment of change in nystagmus intensity as based on a 5-level VRS (0=much worse, 1=worse, 2=unchanged, 3=better, 4=much better)
Patient's subjective change of oscillopsia (MS patients) or nystagmus intensity (CIN patients) as assessed on a 5-level VRS (0=much worse, 1=worse, 2=unchanged, 3=better, 4=much better).
Self-assessment of visual disability (disease-related QoL) by the patient himself/herself, e.g. as based on the Visual Function questionnaire VF14.

### Data Analysis

All efficacy analyses for the CIN subjects is performed on the TPP subset, i.e. all randomized subjects who complete both periods of the study and have no significant protocol deviations as defined by the sponsor prior to unblinding. Descriptive analysis is performed for both the TPP and the FAS subset.

The primary efficacy parameter and all other secondary efficacy parameters regarding Visual Acuity are analyzed using an ANCOVA approach that simultaneously accommodates both between subjects and within subject factors (i.e. a mixed model) with treatment, period and sequence as fixed effects, subject as random effect and baseline value as covariate.

The same model is used for the analysis of change from baseline in nystagmus intensity, NAFX, reading speed, and visual disability measured by the VF-14 questionnaire. For nystagmus intensity at 1.2 m and NAFX the area under the curve (AUC) of the measurement at the null point and the two measurements on either side of the null point is calculated and analyzed in the model described above.

For categorical variables cross-frequency tables are presented per sequence. Tests for treatment effects are performed via a Mainland-Gart test, with non-binary variables (e.g. investigator assessment of change in nystagmus intensity) appropriately dichotomized.

For MS subjects all efficacy analyses are performed descriptively only.

In both patient groups, effects, of treatment are assessed in comparison with placebo using the two-period cross-over design described above (two treatment periods of 7 weeks each, separated by a wash-out phase.

### DISCUSSION

The neramexane treated group demonstrates an improvement in primary outcomes, such as visual acuity, as well as secondary outcomes, such as nystagmus intensity and expanded nystagmus acuity function, reading ability, etc., as compared to the placebo group.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1-20. (canceled)

21. A method of treating nystagmus in a subject in need thereof, comprising administering an effective amount of a 1-aminocyclohexane derivative in a pharmaceutically acceptable carrier.

22. The method of claim 21, wherein the 1-aminocyclohexane derivative is neramexane or a pharmaceutically acceptable salt thereof.

23. The method of claim 22, wherein the 1-aminocyclohexane derivative is neramexane mesylate.

24. The method of claim 21, wherein the 1-aminocyclohexane derivative is administered in a range from about 5 mg to about 150 mg/day.

25. The method of claim 21, wherein the 1-aminocyclohexane derivative is administered in a range from about 5 mg to about 100 mg/day.

26. The method of claim 25, wherein the 1-aminocyclohexane derivative is administered at about 5 mg to about 75 mg/day.

27. The method of claim 26, wherein the 1-aminocyclohexane derivative is administered at about 50 mg/day.

28. The method of claim 26, wherein the 1-aminocyclohexane derivative is administered at about 75 mg/day

29. The method of claim 21, wherein the 1-aminocyclohexane derivative is administered once a day, twice a day (b.i.d.), or three times a day.

30. The method of claim 29, wherein the 1-aminocyclohexane derivative is administered three times a day.

31. The method of claim 30, wherein the 1-aminocyclohexane derivative is administered in an immediate release formulation.

32. The method of claim 21, wherein the 1-aminocyclohexane derivative is administered in a modified release formulation.

33. The method of claim 21, further comprising administration of an additional pharmaceutical agent which has been shown to be effective in treating nystagmus.

34. The method of claim 33, wherein the 1-aminocyclohexane derivative and the additional pharmaceutical agent are administered conjointly.

35. The method of claim 34, wherein the 1-aminocyclohexane derivative and the additional pharmaceutical agent are administered in a single formulation.

36. The method of claim 33, wherein the additional pharmaceutical agent is selected from memantine, baclofen, gabapentin, vigabatrin, pregabalin, 4-aminopyridine, 3,4-diaminopyridine, scopolamine, and clonazepam.

37. The method of claim 21, wherein the therapeutically effective amount of the 1-aminocyclohexane derivative is administered daily for a first period of at least three (3) months, followed by a second period of at least one (1) month wherein the 1-amino-alkylcyclohexane derivative is administered at a dose which is 0-75% of the therapeutically effective dose.

38. The method of claim 37, wherein during the second period the 1-amino-alkylcyclohexane derivative is administered at a dose which is above 0-75% of the therapeutically effective dose, or is administered at a dose which is 20-75% of the therapeutically effective dose, or is administered at a dose which is 25-50% of the therapeutically effective dose.

39. The method of claim 37, wherein during the second period the 1-amino-alkylcyclohexane derivative is not administered with treatment being repeated after the second period.

40. The method of claim 21, wherein the 1-aminocyclohexane derivative is administered in a titration scheme which provides quick and safe attainment of an effective dose.

41. The method of claim 40, wherein the 1-aminocyclohexane derivative is neramexane or a pharmaceutically acceptable salt thereof and is administered according to the following schedule: once daily at a dose of 25 mg per day for the first week, once daily at a dose of 50 mg per day for the second week, and, optionally, once daily at a dose of 75 mg per day for the third week.

42. A pharmaceutical composition comprising a therapeutically effective amount of a 1-aminocyclohexane derivative in combination with an additional pharmaceutical agent which has been shown to be effective for the treatment of nystagmus and, optionally, at least one pharmaceutically acceptable carrier or excipient.

43. The pharmaceutical composition of claim 42, wherein the 1-aminocyclohexane derivative is neramexane or a pharmaceutically acceptable salt thereof.
